(19) United States
(12) Patent Application Publication
(10) Pub. No.: US 2024/0424188 A1
(43) Pub. Date: Dec. 26, 2024

(54) Human Nasal Cleaner

(71) Applicant: Zhiduan ZHANG, Fujian Province (CN)

(72) Inventor: Zhiduan ZHANG, Fujian Province (CN)

(21) Appl. No.: 18/449,131

(22) Filed: Aug. 14, 2023

(30) Foreign Application Priority Data
Jun. 21, 2023 (CN) 202330386534.2
Aug. 3, 2023 (CN) 202322079539.8

(51) Int. Cl. A61M 1/00

(52) U.S. Cl. CPC A61M 1/74

(57) ABSTRACT

The present disclosure provides a human nasal cleaner and relates to a technical field of nasal cleaning devices. The nasal cleaner includes a bottom tank; wherein a cleaning assembly is arranged above the bottom tank to be in contact with a nasal cavity, and a central control assembly is arranged between the bottom tank and the cleaning assembly for transmitting a cleaning liquid; the central control assembly includes a water supply module and a negative pressure generator module, the water supply module including a pumping pipeline, a water pump, and a delivery pipeline, and the negative pressure generator module including a wastewater pipeline and a vacuum pump.

### CROSS REFERENCE

The present disclosure claims priority of Chinese Design Application No. 202330386534.2, filed on Jun. 21, 2023, the entire contents of which are hereby incorporated by reference in their entirety.

The present disclosure claims priority of Chinese Utility Model Application No. 202322079539.8, filed on Aug. 3, 2023, the entire contents of which are hereby incorporated by reference in their entirety.

### TECHNICAL FIELD

The present disclosure relates to the technical field of nasal cleaning devices, and specifically relates to a human nasal cleaner.

### BACKGROUND

According to incomplete statistics, there are about 200 million rhinitis patients in China. Most of the rhinitis takes a long period of treatment but is not cured, which plagues people's lives and health over and over again. Therefore, attention to the nasal cavity is particularly important. Most of the nasal cleaning products on the market are simple, and three main types thereof are: a simplest type requiring manual pressing to push water, a type with functions of automatic water-filling and one-sided vacuum suction, and a type with electric water-filling function.

That is, when the existing devices for cleaning the nasal cavity are used, the nasal cavity is filled with a large amount of cleaning liquid, which may cause discomfort in the patient's nasal cavity and even choking.

For this reason, an improved human nasal cleaner is proposed.

### SUMMARY OF THE DISCLOSURE

The purpose of the present disclosure is to solve the problems existing in the related art and provide a human nasal cleaner.

The present disclosure specifically adopts the following technical solutions in order to realize the above purposes.

A nasal cleaner, including a bottom tank; wherein a cleaning assembly is arranged above the bottom tank to be in contact with a nasal cavity, and a central control assembly is arranged between the bottom tank and the cleaning assembly for transmitting a cleaning liquid; the central control assembly includes a water supply module and a negative pressure generator module, the water supply module including a pumping pipeline, a water pump, and a delivery pipeline, and the negative pressure generator module including a wastewater pipeline and a vacuum pump.

In some embodiments, an inner cavity of the bottom tank is divided by a partition into a clean water bin for storing a clean cleaning liquid and a wastewater bin for storing a waste liquid after nasal cleaning.

In some embodiments, the cleaning assembly includes a water supply hole and a drainage hole; an output end of the delivery pipeline is connected to the water supply hole for discharging the clean cleaning liquid into the nasal cavity for the nasal cleaning, and the wastewater bin is connected to the wastewater pipeline for conveying the waste liquid after the nasal cleaning; an input end of the wastewater pipeline is connected to an output end of the drainage hole.

In some embodiments, the wastewater bin is sealed and fixed with a pumping end of the vacuum pump for changing a pressure inside the wastewater bin.

In some embodiments, the drainage hole, the wastewater bin, and the wastewater pipeline are connected and sealed by hot-melt adhesion.

In some embodiments, an inner cavity of the clean water bin is placed with the pumping pipeline for conveying the clean cleaning liquid, the water pump is connected to an output end of the pumping pipeline, and an outlet end of the water pump is connected to an input end of the water supply hole through the delivery pipeline.

Beneficial effect is as follows.

1. the water supply module facilitates pumping the clean cleaning liquid stored in the inner cavity of the clean water bin to the patient's nasal cavity for rinsing, and at the same time, the negative pressure generator module can be driven to adjust the pressure in the inner cavity of the wastewater bin to a negative pressure to facilitate suctioning the waste liquid from the other side of the nasal cavity to the inner cavity of the wastewater bin. The design of negative-pressure water suction while pressurized water supply, with equal amount of water, can ensure that the cleaning liquid inside the nasal cavity flows smoothly when in use, thereby effectively preventing choking accidents from occurring and enhancing the patient's sense of usage comfort.

2. the vacuum pump can suck the waste liquid after cleaning into the wastewater bin, which effectively ensures that the cleaning liquid inside the nasal cavity can flow smoothly, avoids discomfort in the patient's nasal cavity caused by the nasal cavity being filled with a large amount of cleaning liquid, and effectively prevents choking accidents from occurring.

3. the water pump is arranged such that clean cleaning liquid from the inner cavity of the clean water bin can be conveyed into the delivery pipeline, and the clean cleaning liquid can be conveyed into the nasal cavity through the water supply hole for nasal cleaning.

### BRIEF DESCRIPTION OF THE DRAWINGS

FIG. 1 is a perspective view of an overall structure of a human nasal cleaner according to the present disclosure.

FIG. 2 is a structural schematic view of a central control assembly according to the present disclosure.

### REFERENCE NUMERALS

1, bottom tank; 11, clean water bin; 12, wastewater bin; 2, center control assembly; 21, pumping pipeline; 22, water pump; 23, delivery pipeline; 24, wastewater pipeline; 25, vacuum pump; 3, cleaning assembly; 31, water supply hole; 32, drainage hole.

### DETAILED DESCRIPTION

In order to make the purpose, technical solutions, and advantages of the present disclosure embodiments clearer, the technical solutions in the embodiments will be clearly and completely described below in conjunction with the accompanying drawings in the present disclosure. It is clear that the described embodiments are a part of the embodiments of the present disclosure and not all of the embodiments. Generally, the components of the embodiments of the present disclosure described and illustrated in the accompanying drawings herein can be arranged and designed in a variety of different configurations.

Accordingly, the following detailed description of the embodiments of the present disclosure provided in the accompanying drawings is not intended to limit the scope of the present disclosure, but rather represents only selected embodiments of the present disclosure. Based on the embodiments in the present disclosure, all other embodiments obtained by those skilled in the art without creative labor fall within the scope of the present disclosure.

It should be noted that similar symbols and letters denote similar items in the following accompanying drawings, and therefore, once an item is defined in one of the accompanying drawings, it does not need to be further defined and explained in the subsequent accompanying drawings. Furthermore, the terms “first”, “second”, etc. are intended only to distinguish descriptions and are not to be understood as indicating or implying relative importance.

In the description of the embodiments of the present disclosure, it is to be clarified that the orientation or positional relationship indicated by the terms “inner”, “outer”, “upper”, and the like is based on the orientation or positional relationship shown in the accompanying drawings, or the orientation or positional relationship in which the product is customarily placed when used, which is only for the purpose of facilitating the description of the present disclosure and simplifying the description, but not intended to indicate or imply that the device or element referred to must be of a particular orientation or constructed and operated with a particular orientation, and therefore not to be construed as limitations on the present disclosure.

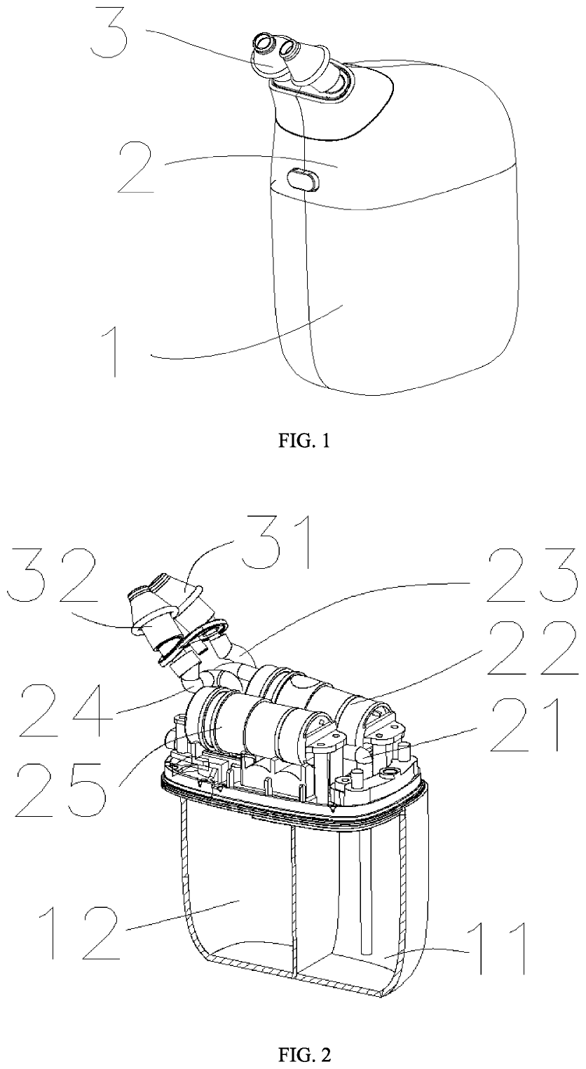

Embodiment 1: referring to FIGS. 1 to 2, a human nasal cleaner includes a bottom tank 1; where a cleaning assembly 3 is arranged above the bottom tank 1 to be in contact with a human nasal cavity, and a central control assembly 2 is arranged between the bottom tank 1 and the cleaning assembly 3 for transmitting a cleaning liquid; the central control assembly 2 includes a water supply module and a negative pressure generator module. The water supply module includes a pumping pipeline 21, a water pump 22, a delivery pipeline 23. The negative pressure generator module includes a wastewater pipeline 24 and a vacuum pump 25.

In order to facilitate the classification and collection of the cleaning liquid before and after use, an inner cavity of the bottom tank 1 is divided by means of a partition into a clean water bin 11 for storing a clean nasal cleaning liquid and a wastewater bin 12 for storing a waste liquid after the nasal cleaning.

Specifically, the water supply module facilitates pumping the clean cleaning liquid stored in the inner cavity of the clean water bin 11 to the patient's nasal cavity for rinsing, and at the same time, the negative pressure generator module can be driven to adjust the pressure in the inner cavity of the wastewater bin 12 to a negative pressure to facilitate suctioning the waste liquid from the other side of the nasal cavity to the inner cavity of the wastewater bin 12. The design of negative-pressure water suction while pressurized water supply, with equal amount of water, can ensure that the cleaning liquid inside the nasal cavity flows smoothly when in use, thereby effectively preventing choking accidents from occurring and enhancing the patient's sense of usage comfort.

When using the nasal cleaner, the patient can insert the cleaning assembly 3 into the nasal cavity and synchronously drive the water pump 22 and the vacuum pump 25, such that the water pump 22 can pump the clean nasal cleaning liquid stored inside the clean water bin 11 through the pumping pipeline 21. The cleaning liquid sucked out from the pumping pipeline 21 is transported to a side of the nasal cavity to be cleaned through the delivery pipeline 23, and at the same time, the vacuum pump 25 is driven to cause the internal cavity of the wastewater bin 12 to produce negative pressure. The negative pressure can be spread to the patient's nasal cavity, such that the cleaning liquid after cleaning the nasal cavity can be sucked from the other side of the nasal cavity to the inner cavity of the wastewater pipeline 24 and then transported to the inner cavity of the wastewater bin 12 through the wastewater pipeline 24 for uniform collection and treatment, thereby effectively ensuring that the cleaning liquid inside the nasal cavity can flow smoothly. In one nostril, water is pumped in to provide water for cleaning the nasal cavity; and in the other nostril, negative pressure is adopted to suck out water with equal amount, which can achieve comfortable cleaning and anti-choking effect.

Embodiment 2: based on Embodiment 1, referring to FIGS. 1 to 2, the cleaning assembly 3 includes a water supply hole 31 and a drainage hole 32. An output end of the delivery pipeline 23 is connected to the water supply hole 31 for discharging the cleaning liquid into the nasal cavity for cleaning, and the wastewater bin 12 is connected to the wastewater pipeline 24 for conveying the waste liquid after cleaning of the nasal cavity. An input end of the wastewater pipeline 24 is connected to an output end of the drainage hole 32.

To facilitate the change of the pressure of the inner cavity of the wastewater bin 12, the wastewater bin 12 is sealed and fixed with a pumping end of the vacuum pump 25 for changing the pressure inside the wastewater bin 12.

To enhance the sealing of the connection between the drainage hole 32, the wastewater bin 12, and the wastewater pipeline 24, the drainage hole 32, the wastewater bin 12, and the wastewater pipeline 24 are all connected and sealed by means of hot-melt adhesion.

Specifically, the vacuum pump 25 can suck the waste liquid after cleaning into the wastewater bin 12, which effectively ensures that the cleaning liquid inside the nasal cavity can flow smoothly, avoids discomfort in the patient's nasal cavity caused by the nasal cavity being filled with a large amount of cleaning liquid, and effectively prevents choking accidents from occurring.

When using the nasal cleaner, the patient can insert the water supply hole 31 and the drainage hole 32 into the two nostrils, and the vacuum pump 25 is driven to change the internal pressure of the wastewater bin 12 to a negative pressure, such that the waste liquid after cleaning the nasal cavity can be sucked into the wastewater bin 12 through the drainage hole 32 for uniform collection and treatment.

Embodiment 3: based on Embodiment 1, referring to FIG. 2, the inner cavity of the clean water bin 11 is placed with a pumping pipeline 21 for conveying the cleaning liquid, a water pump 22 is connected to an output end of the pumping pipeline 21, and an outlet end of the water pump 22 is connected to an input end of the water supply hole 31 through the delivery pipeline 23.

Specifically, the water pump 22 is arranged such that clean cleaning liquid from the inner cavity of the clean water bin 11 can be conveyed into the delivery pipeline 23, and the clean cleaning liquid can be conveyed into the nasal cavity through the water supply hole 31 for nasal cleaning.

When using the nasal cleaner, the patient can first insert the water supply hole 31 and the drainage hole 32 into the two nostrils, and the water pump 22 is driven to pump the clean cleaning liquid from the inner cavity of the clean water bin 11 into the delivery pipeline 23 through the pumping pipeline 21, such that the clean cleaning liquid can be transported to a side of the nasal cavity through the water supply hole 31 for nasal cleaning.

In summary: the water supply module facilitates pumping the clean cleaning liquid stored in the internal cavity of the clean water bin 11 to a side of the patient's nasal cavity, for rinsing the patient's nasal cavity. At the same time, the negative pressure generator module is driven to adjust the pressure in the internal cavity of the wastewater bin 12 to negative for sucking the waste liquid from the other side of the nasal cavity to the internal cavity of the wastewater bin 12. The design of negative-pressure water suction while pressurized water supply, with equal amount of water, can ensure that the cleaning liquid inside the nasal cavity flows smoothly when in use, thereby effectively preventing choking accidents from occurring and enhancing the patient's sense of usage comfort.

The vacuum pump 25 can suck the waste liquid after cleaning into the wastewater bin 12, which effectively ensures that the cleaning liquid inside the nasal cavity can flow smoothly, avoids discomfort in the patient's nasal cavity caused by the nasal cavity being filled with a large amount of cleaning liquid, and effectively prevents choking accidents from occurring.

The water pump 22 is arranged such that clean cleaning liquid from the inner cavity of the clean water bin 11 can be conveyed into the delivery pipeline 23, and the clean cleaning liquid can be conveyed into the nasal cavity through the water supply hole 31 for nasal cleaning.

The above shows and describes the basic principle, the main features, and the advantages of the present disclosure. Those skilled in the art should understand that the present disclosure is not limited by the above embodiments, and that what is described in the above embodiments and the specification is only the principle of the present disclosure, and that there will be various changes and improvements to the present disclosure without departing from the spirit and scope of the present disclosure, and that all of these changes and improvements fall within the scope of the present disclosure. The scope of the present disclosure is defined by the appended claims and their equivalents.

What is claimed is:

1. A human nasal cleaner, comprising a bottom tank (1); wherein a cleaning assembly (3) is arranged above the bottom tank (1) to be in contact with a nasal cavity, and a central control assembly (2) is arranged between the bottom tank (1) and the cleaning assembly (3) for transmitting a cleaning liquid; the central control assembly (2) comprises a water supply module and a negative pressure generator module, the water supply module comprising a pumping pipeline (21), a water pump (22), and a delivery pipeline (23), and the negative pressure generator module comprising a wastewater pipeline (24) and a vacuum pump (25).

2. The human nasal cleaner according to claim 1, wherein an inner cavity of the bottom tank (1) is divided by a partition into a clean water bin (11) for storing a clean cleaning liquid and a wastewater bin (12) for storing a waste liquid after nasal cleaning.

3. The human nasal cleaner according to claim 2, wherein the cleaning assembly (3) comprises a water supply hole (31) and a drainage hole (32); an output end of the delivery pipeline (23) is connected to the water supply hole (31) for discharging the clean cleaning liquid into the nasal cavity for the nasal cleaning, and the wastewater bin (12) is connected to the wastewater pipeline (24) for conveying the waste liquid after the nasal cleaning; an input end of the wastewater pipeline (24) is connected to an output end of the drainage hole (32).

4. The human nasal cleaner according to claim 2, wherein the wastewater bin (12) is sealed and fixed with a pumping end of the vacuum pump (25) for changing a pressure inside the wastewater bin (12).

5. The human nasal cleaner according to claim 3, wherein the drainage hole (32), the wastewater bin (12), and the wastewater pipeline (24) are connected and sealed by hot-melt adhesion.

6. The human nasal cleaner according to claim 3, wherein an inner cavity of the clean water bin (11) is placed with the pumping pipeline (21) for conveying the clean cleaning liquid, the water pump (22) is connected to an output end of the pumping pipeline (21), and an outlet end of the water pump (22) is connected to an input end of the water supply hole (31) through the delivery pipeline (23).
